(19) United States
(12) Patent Application Publication
(10) Pub. No.: US 2024/0065874 A1
(43) Pub. Date: Feb. 29, 2024

(54) FLUID COLLECTION ASSEMBLIES INCLUDING AT LEAST ONE NONWOVEN MATERIAL

(71) Applicant: PureWick Corporation, Covington, GA (US)

(72) Inventors: Seth Schneider, Social Circle, GA (US); Anthony Esposito, Oxford, GA (US); Samuel Hernandez, Covington, GA (US); Simon Ofosu, Lilburn, GA (US); Robert Hine, Covington, GA (US)

(21) Appl. No.: 18/259,626

(22) PCT Filed: Jan. 5, 2022
(86) PCT No.: PCT/US2022/011281

§ 371 Date: Jun. 28, 2023

Related U.S. Application Data
(60) Provisional application No. 63/134,754, filed on Jan. 7, 2021.

(51) Int. Cl. A61F 5/44; A61F 5/453

(52) U.S. Cl. CPC A61F 5/4401; A61F 5/453

(57) ABSTRACT

Embodiments disclosed herein include fluid collection assemblies having at least one nonwoven material, fluid collection systems including the same, and methods of using the same. An example fluid collection assembly includes a fluid impermeable barrier. The fluid impermeable barrier defines a chamber, at least one opening, and a fluid outlet. The fluid collection assembly also include at least one porous material disposed in the chamber. The porous material includes at least one nonwoven material.

### CROSS-REFERENCE TO RELATED APPLICATION(S)

This application claims priority to U.S. Provisional Patent Application No. 63/134,754 filed on Jan. 7, 2021, the disclosure of which is incorporated herein, in its entirety, by this reference.

### BACKGROUND

A patient may have limited or impaired mobility such that typical urination processes are challenging or impossible. For example, the patient may have surgery or a disability that impairs mobility. In another example, the patient may have restricted travel conditions such as those experience by pilots, drivers, and workers in hazardous areas. Additionally, fluid collection from the patient may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections.

Thus, users and manufacturers of fluid collection assemblies continue to seek new and improved devices, systems, and methods to collect urine.

### SUMMARY

Embodiments disclosed herein include fluid collection assemblies having at least one nonwoven material, fluid collection systems including the same, and methods of using the same. An example fluid collection assembly includes a fluid impermeable barrier. In an embodiment, a fluid collection assembly. The fluid collection assembly includes a fluid impermeable barrier at least defining a chamber, at least one opening, and a fluid outlet. The fluid collection assembly also includes at least one porous material disposed in the chamber. The at least one porous material includes at least one nonwoven material.

In an embodiment, a fluid collection system is disclosed. The fluid collection system includes a fluid storage container configured to hold one or more bodily fluids therein. The fluid collection system also includes a fluid collection assembly. The fluid collection assembly includes a fluid impermeable barrier at least defining a chamber, at least one opening, and a fluid outlet. The fluid collection assembly also includes at least one porous material disposed in the chamber. The at least one porous material includes at least one nonwoven material. The fluid collection system further includes a vacuum source in fluid communication with the fluid storage container and the fluid collection assembly. The vacuum source is configured to draw the one or more bodily fluids from the fluid collection assembly and deposit the one or more bodily fluids in the fluid storage container via one or more conduits.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection assembly, according to an embodiment.

FIG. 1B is a cross-sectional view of the fluid collection assembly taken along plane 1B-1B shown in FIG. 1A.

FIG. 2 is a cross-sectional schematic of a fluid collection assembly, according to an embodiment.

FIG. 3 is a cross-sectional schematic of a fluid collection assembly, according to an embodiment.

FIG. 4A is an isometric view of a fluid collection assembly, according to an embodiment.

FIG. 4B is a cross-sectional schematic of the fluid collection assembly taken along plane 4B-4B shown in FIG. 4A, according to an embodiment.

FIG. 5 is a block diagram of a system for fluid collection, according to an embodiment.

### DETAILED DESCRIPTION

Embodiments disclosed herein include fluid collection assemblies having at least one nonwoven material, fluid collection systems including the same, and methods of using the same. An example fluid collection assembly includes a fluid impermeable barrier. The fluid impermeable barrier defines a chamber, at least one opening, and a fluid outlet. The fluid collection assembly also include at least one porous material disposed in the chamber. The porous material includes at least one nonwoven material. Nonwoven materials include a plurality of fibers that have been bonded together using at least one of heat, chemical (e.g., adhesive or other binder), mechanical (e.g., entanglement), or other treatment. Examples of nonwoven materials includes carded webs, needle punched webs, air laid webs, spunlace webs, vertical lapped nonwoven fabrics, horizontal lapped nonwoven fabrics, and crossed lapped nonwoven fabric.

During use, the fluid collection assembly may be positioned such that the opening is positioned adjacent to a female urethral opening or receive a male urethral opening (e.g., penis). The fluid collection assembly may receive one or more bodily fluids (e.g., urine, blood, sweat, etc.) into the porous material. The porous material may move the bodily fluids received thereby towards the fluid outlet. A vacuum pressure may be applied to the chamber from the fluid outlet. The vacuum pressure may remove the bodily fluids from the chamber, for example, via a conduit and deposit the bodily fluids in a fluid collection assembly.

Conventional fluid collection assemblies (i.e., fluid collection assemblies without nonwoven materials) often include woven materials disposed in the chambers thereof. Woven materials are materials formed by weaving multiple threads together. The woven material are often used to absorb, wick, temporarily store, or otherwise receive one or more bodily fluids therein. Examples of woven materials include gauze and spun nylon fibers. Woven materials may be expensive. Woven materials may be susceptible to collapse when a vacuum pressure is applied to the fluid collection assembly, for example, when the fluid impermeable barrier of the fluid collection assembly cannot resist the vacuum pressure without collapsing. The collapse of the woven material may impede the flow of the bodily fluids through the woven materials and, when the fluid impermeable barrier also collapses, may prevent the vacuum pressure from being applied to portions of the fluid collection assembly upstream from the collapse. The woven materials may also have issues moving the bodily fluids towards the fluid outlet. For example, spun nylon fibers may merely provide air space to store the bodily fluids and, if not for a pressure differential and gravity, may not move significant quantities of the bodily fluids towards the fluid outlet. Other woven materials may only move the bodily fluids substantially in a horizontal direction (e.g., parallel to length and/or width thereof) or a vertical direction (e.g., parallel to the thickness). Woven materials requires long fibers (e.g., fibers exhibiting a length greater than 25 cm) and may only be formed from certain fibers (e.g., solid fibers), both of which reduces the customization of the woven materials.

As previously discussed, the fluid collection assemblies disclosed herein include at least one nonwoven material instead of or in addition to at least one woven material. The nonwoven materials disclosed herein are an improvement over at least some woven materials. For example, nonwoven materials may be cheaper than woven materials. The nonwoven materials may be manufactured with high loft (i.e., thickness) and strength which may prevent the collapse of the nonwoven material even when a strong vacuum pressure is applied to the chamber and the fluid impermeable barrier would otherwise collapse. The nonwoven materials may also be able to wick the bodily fluids therethrough in both horizontal and vertical directions. Nonwoven materials may also be formed from fibers that cannot be used in woven materials, such as relatively short fibers (e.g., fibers exhibiting a length less than 10 cm) and from materials that cannot be used in woven materials (e.g., rigid or hollow fibers). Forming the nonwoven materials from fibers that cannot be used in woven materials allows for the nonwoven materials exhibiting a wider variety of properties to be used in the fluid collection assemblies disclosed herein than the convention fluid collection assemblies.

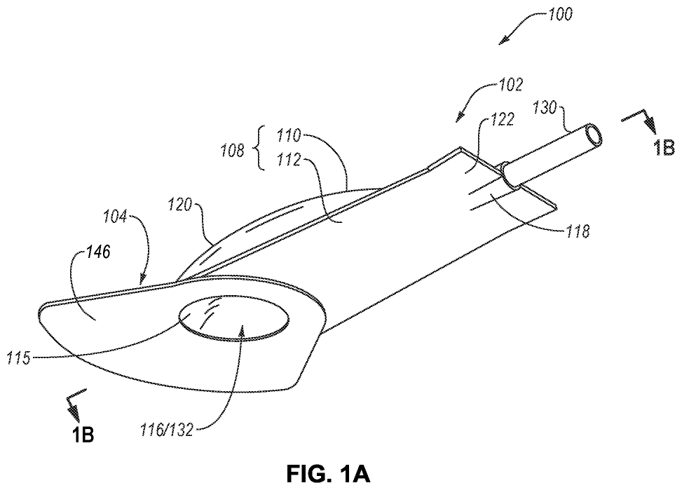

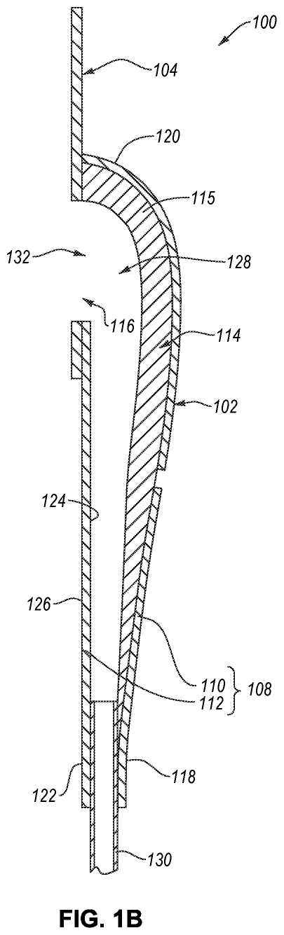

FIG. 1A is an isometric view of a fluid collection assembly 100, according to an embodiment. FIG. 1B is a cross-sectional view of the fluid collection assembly 100 taken along plane 1B-1B shown in FIG. 1A. The fluid collection assembly 100 is an example of a male fluid collection assembly that is configured to collect one or more bodily fluids from a male urethral opening (e.g., penis). However, it is noted that the fluid collection assembly 100 may also be used to collection bodily fluids from a female urethral opening. The fluid collection assembly 100 includes a sheath 102 and a base 104. The base 104 is configured to be attached (e.g., permanently attached to or configured to be permanently attached) to the sheath 102. The base 104 is also configured to be attached to the region about the urethral opening (e.g., penis) of the patient.

The sheath 102 includes a fluid impermeable barrier 108 that is at least partially formed from a first panel 110 and a second panel 112. The first panel 110 and the second panel 112 may be attached or integrally formed together (e.g., exhibits single piece construction). In an embodiment, as illustrated, the first panel 110 and the second panel 112 are distinct sheets. The fluid impermeable barrier 108 also defines a chamber 114 between the first panel 110 and the second panel 112, at least one opening 116 at a first end region 120 of the sheath 102, and an fluid outlet 118 at a second end region 122 of the sheath 102. The sheath 102 also includes at least one porous material 115 disposed in the chamber 114.

The inner surface(s) 124 of the fluid impermeable barrier 108 (e.g., inner surfaces of the first and second panels 110, 112) at least partially defines the chamber 114 within the fluid collection assembly 100. The fluid impermeable barrier 108 temporarily stores the bodily fluids in the chamber 114. The fluid impermeable barrier 108 may be formed of any suitable fluid impermeable material(s), such as a fluid impermeable polymer (e.g., silicone, polypropylene, polyethylene, polyethylene terephthalate, neoprene, a polycarbonate, etc.), a metal film, natural rubber, another suitable material, or combinations thereof. As such, the fluid impermeable barrier 108 substantially prevents the bodily fluids from passing through the fluid impermeable barrier 108. In an example, the fluid impermeable barrier 108 may be air permeable and fluid impermeable. In such an example, the fluid impermeable barrier 108 may be formed of a hydrophobic material that defines a plurality of pores. At least one or more portions of at least an outer surface 126 of the fluid impermeable barrier 108 may be formed from a soft and/or smooth material, thereby reducing chaffing. The first and second panels 110, 112 may be formed from films (e.g., exhibit a thickness that is less than about 1 mm or less than 0.5 mm). Forming the first and second panels 110, 112 as films may facilitate the sheath 102 lying flat but increases the likelihood that the first and second panels 110, 112 would collapse due the vacuum pressure if not form the porous material 115.

In an embodiment, at least one of the first panel 110 or the second panel 112 is formed from an at least partially transparent fluid impermeable material, such as polyethylene, polypropylene, polycarbonate, or polyvinyl chloride. Forming at least one of the first panel 110 or the second panel 112 from an at least partially transparent fluid impermeable material allows a person (e.g., medical practitioner) to examiner the penis. In some embodiments, both the first panel 110 and the second panel 112 are formed from at least partially transparent fluid impermeable material. Selecting at least one of the first panel 110 or the second panel 112 to be formed from an at least partially transparent impermeable material allows the penis to be examined without detaching the entire fluid collection assembly 100 from the region about the penis. For example, the chamber 114 may include a penis receiving area 128 that is configured to receive the penis of the individual when the penis extends into the chamber 114. The penis receiving area 128 may be defined by at least the porous material 115 and at least a portion of the at least partially transparent material of the first panel 110 and/or the second panel 112. In other words, the porous material 115 is positioned in the chamber 114 such that the porous material 115 is not positioned between the penis and at least a portion of the transparent portion of the first panel 110 and/or second panel 112 when the penis is inserted into the chamber 114 through the opening 116. The porous material 115 is generally not transparent and, thus, the portion of the at least partially transparent material of the first panel 110 and/or the second panel 112 that defines the penis receiving area 128 forms a window which allows the person to view into the penis receiving area 128 and examine the penis.

The opening 116 defined by the fluid impermeable barrier 108 provides an ingress route for fluids to enter the chamber 114 when the penis is a buried penis and allow the penis to enter the chamber 114 (e.g., the penis receiving area 128) when the penis is not buried. The opening 116 may be defined by the fluid impermeable barrier 108 (e.g., an inner edge of the fluid impermeable barrier 108). For example, the opening 116 is formed in and extends through the fluid impermeable barrier 108 thereby enabling bodily fluids to enter the chamber 114 from outside of the fluid collection assembly 100.

The fluid impermeable barrier 108 defines an fluid outlet 118 sized to receive an conduit 130. The conduit 130 may be at least partially disposed in the chamber 114 or otherwise in fluid communication with the chamber 114 through the fluid outlet 118. The fluid outlet 118 may be sized and shaped to form an at least substantially fluid tight seal against the conduit 130 thereby substantially preventing the bodily fluids from escaping the chamber 114. In an embodiment, the fluid outlet 118 may be formed from a portion of the first panel 110 and the second panel 112 that are not attached or integrally formed together. In such an embodiment, the fluid impermeable barrier 108 may not include a cap exhibiting a rigidity that is greater than the portions of the fluid impermeable barrier 108 thereabout which may facilitate manufacturing of the fluid collection assembly 100 may decreasing the number of parts that are used to form the fluid collection assembly 100 and may decrease the time required to manufacture the fluid collection assembly 100. The lack of the cap may make securing the conduit 130 to the fluid outlet 118 using interference fit to be difficult though, it is noted, attaching the conduit 130 to the fluid outlet 118 may still be possible. As such, the conduit 130 may be attached to the fluid outlet 118 (e.g., to the first and second panels 110, 112) using an adhesive, a weld, or otherwise bonding the fluid outlet 118 to the fluid outlet 118. Attaching the conduit 130 to the fluid outlet 118 may prevent leaks and may prevent the conduit 130 from inadvertently becoming detached from the fluid outlet 118. In an example, the conduit 130 may be attached to the fluid outlet 118 in the same manufacturing step that attaches the first and second panels 110, 112 together. In an embodiment, the fluid collection assembly 100 includes a cap defining the fluid outlet 118 that is attached to the fluid impermeable barrier 108. The cap may exhibit a rigidity that is greater than the portion of the fluid impermeable barrier 108 thereabout.

As previously discussed, the sheath 102 includes at least one porous material 115 disclosed in the chamber 114. The porous material 115 may direct the bodily fluids to one or more selected regions of the chamber 114, such as away from the penis and towards the fluid outlet 118. The porous material 115 includes at least one nonwoven material. As previously discussed, the nonwoven material is formed from a plurality of fibers that are bonded together using at least one of heat, chemical, mechanical, or other bonding techniques. The nonwoven material may exhibit at least one of a density that is less than, a thickness that is greater than, a basis weight that is less than, or a fiber length that is less than a woven material which may improve the functionality of the nonwoven material relative to woven materials. For example, as previously discussed, the first and second panels 110, 112 may be films that are unable, by themselves, to prevent collapse when a vacuum pressure is applied to the chamber 114. However, the nonwoven material may exhibit one or more properties (e.g., strength, density, basis weight, or thickness) that prevents the collapse of the nonwoven material. Further, the nonwoven material may hold the first and second panels 110, 112 apart when the vacuum pressure is applied to the chamber 114.

The nonwoven material exhibits a length, a width that is less than the length, and a thickness that is less than the width. As used herein, a horizontal direction refers to a direction that is generally in-plane with the length and the width (e.g., generally parallel to the length and/or the width) and a vertical direction refers to a direction that is generally parallel to the thickness. The nonwoven materials disclosed herein are able to wick the bodily fluids in at least one of a horizontal direction or a vertical direction without the need of the vacuum pressure and without saturation of the nonwoven material, unlike at least some woven materials such as gauze and spun nylon. Examples of nonwoven materials that may wick the bodily fluids in both the vertical and horizontal directions include vertical lapped nonwoven fabrics, carded webs (e.g., thermally-bonded carded webs), spunlace web, needle punched webs, or a blend or combination thereof.

The nonwoven material may include any suitable nonwoven material. In an embodiment, the nonwoven material includes at least one carded web. The carded web includes a plurality of fibers that may be generally oriented in the same direction. The generally same orientation of the fibers of the carded web cause the carded web to be anisotropic. For example, the strength of the carded web is greatest when a force applied thereto is generally parallel to the fibers but the strength of the carded web decreases as the force applied thereto becomes more oblique or perpendicular to the orientation of the fibers. As such, the carded web may need to be positioned in the chamber 114 to mitigate forces being applied to the carded web that are not generally parallel to the orientation of the fibers or requires addition binding between the fibers (e.g., heat or chemical) to prevent unsatisfactory wear of the carded web. The flow the bodily fluids through the carded web may vary depending on whether the bodily fluids are flowing parallel, obliquely, or perpendicular to the orientation of the fibers. As such, selecting the nonwoven material to include the carded web allows for selecting the strength and flow characteristics of the porous material 115 based on the orientation of the fibers. Even though the fibers are generally oriented, the orientation of each of the fibers may slightly vary which causes the porosity of the carded web to be sufficiently high that the carded web exhibits any of the density, thickness, basis weight, and flow rates disclosed herein.

In an embodiment, the nonwoven material may include at least one needle punched web. The needle punched web may be formed from a sheet including a plurality of fibers. The sheet may include a plurality of randomly oriented fibers (e.g., fibers are generally parallel to and randomly oriented in the horizontal direction), or a carded web since the orientation of the fibers may better facilitate flow of the bodily fluids therethrough. A plurality of needles (e.g., a plurality of barbed needles) are inserted into the sheet in a direction that is generally parallel to a thickness of the sheet which causes some of the fibers to become entangled and interlocked. For example, insertion of the needles into the sheet cause some of the fibers to reorient and migrate from the surface of the sheet to an interior thereof to form columns. The entanglement of the fibers caused by the insertion of the needles may sufficiently entangle the fibers such that no additional binding may be necessary to bond the fibers together. The entanglement of the fibers may cause the needle punched web to exhibit more isotropic properties compared to the carded web and, thus, may not require specific orientation in the chamber 114 or additional binding of the fibers. The needle punched web may exhibit good flow features. For example, the needles extending into the sheet may form divots which facilitate flow of the bodily fluids vertically through the needle punched web.

In an embodiment, the nonwoven material may include at least one air laid web. The air laid web may exhibit a plurality of randomly oriented fibers (e.g., fibers are generally parallel to and randomly oriented in the horizontal direction). The plurality of random fibers may exhibit a length that is sufficiently large that the fibers become entangled and do not need be bounded together or the fibers may be bonded. Due to the random orientation of the fibers, the air laid web tends to be isotropic and exhibit a high porosity. Similar, due to the random orientation of the fibers, the air laid web may exhibit a high loft. The air laid web may be formed from fibers that cannot be carded (e.g., short fibers).

In an embodiment, the nonwoven material may include at least one spunlace web. The spunlace web is formed by providing a sheet that includes randomly oriented fibers or a carded web. High pressure water jets that are generally parallel to the thickness of the sheet are directed towards the sheet. Similar to the needle punched web, the high pressure jets of water cause some of the fibers to migrate from an exterior of the sheet to an interior thereof to form columns Thus, the spunlace web may function similar to the needle punched web, namely that the spunlace web may be more isotropic than the carded web and includes divots. However, the spunlace web may exhibit at least one of a density that is less than, a thickness that is greater than, or a base weight that is less than the needle punched web. As such, the spunlace web may be more delicate (e.g., less durable or softer) than the needle punched web. The more delicate spunlace web may more comfortably contact the skin of the patient than the needle punched web.

In an embodiment, the nonwoven web may include at least one vertical lapped nonwoven fabric. The vertical lapped nonwoven fabric is formed by lapping a sheet vertically such that a cross-section of the vertical lapped nonwoven fabric taken along a plane that is parallel to the thickness and length of the vertical lapped nonwoven fabric shows a periodic wavy (e.g., sinusoidal) structure. The folds in the sheet cause the fibers of the nonwoven web to be preferentially oriented vertically between the folds and the fibers at the folds to be preferentially oriented horizontally. Thus, the vertically lapped nonwoven fabric causes the bodily fluids to wick both horizontally and vertically. The vertically oriented fibers also cause the vertical lapped nonwoven fabric to be resistant to collapse, even at high vacuum pressures. Similarly, the vertically oriented fibers also cause the vertical lapped nonwoven fabric to exhibit excellent elastic recovery and localized deformation when a force is applied thereto that is generally parallel to the vertically oriented fibers. The elastic recovery and localized deformation minimize the likelihood that the nonwoven material collapses when both a vacuum pressure and an external force is applied to the nonwoven web (e.g., laying on or pressing against fluid collection assembly 100) and increases the likelihood that any collapse is localized and temporary. The vertical lapped nonwoven fabric may also exhibit low density and is highly moldable. The vertical lapped nonwoven fabric may exhibit any suitable thickness by increasing or decreasing the distance between folds.

In an embodiment, the nonwoven web may include at least one horizontal lapped nonwoven fabric. The horizontal lapped nonwoven fabric is formed by lapping a sheet horizontally. The folds in the sheet cause the fibers of the nonwoven web to be preferentially oriented horizontally between the folds and the fibers at the folds to be preferentially oriented vertically. Thus, the horizontal lapped nonwoven fabric causes the bodily fluids to wick both horizontally and vertically. The horizontal lapped nonwoven fabric may exhibit high thickness by merely increasing the number of folds formed therein.

In an embodiment, the nonwoven web may include at least one crossed lapped nonwoven fabric. The crossed lapped nonwoven fabric is substantially similar to the horizontal lapped nonwoven fabric except that each layer is not parallel to the adjacent layers. Instead, each layer extends obliquely relative to the previously layer which causes the crossed lapped nonwoven fabric to exhibit more isotropic properties than the horizontal lapped nonwoven web, especially when the horizontal and crossed lapped nonwoven fabrics are formed from a carded web.

It is currently believed that the carded web, needle punched web, the air laid web, the spunlace web, the vertical lapped nonwoven fabric, the horizontal lapped nonwoven fabric, and the crossed lapped nonwoven fabric are the preferred nonwoven materials to be included in the porous material 115. However, it is noted that the porous material 115 may include one or more nonwoven materials other than the carded web, needle punched web, the air laid web, the spunlace web, the vertical lapped nonwoven fabric, the horizontal lapped nonwoven fabric, and the crossed lapped nonwoven. In an example, the nonwoven material may include a wet laid web even though the wet laid web may exhibit low durability compared to the other nonwoven materials disclosed herein. In an example, the nonwoven material may include spunbonded or meltblow nonwovens even though such nonwovens may exhibit too low of porosity for some applications.

In an embodiment, the nonwoven material of the porous material 115 may be selected based on the expected vacuum pressure that is applied to the chamber 114 such that the nonwoven material is unlikely to collapse when exposed to the vacuum pressure. As used herein, the vacuum pressure refers to the absolute pressure or the gauge pressure. The absolute pressure is the pressure differential between a vacuum and a location within a portion of the conduit 130 that is adjacent to the chamber 114. The gauge pressure is the pressure differential between a location external to and spaced from the fluid collection assembly 100 (e.g., about 101 kPa) and a location within a portion of the conduit 130 that is adjacent to the chamber 114. During use, the vacuum pressure applied to the chamber 114 may be about 5 kPa to about 40 kPa depending on the vacuum source that is fluidly coupled to the fluid collection assembly 100. As such, the nonwoven material of the porous material 115 may be selected to be able to withstand such vacuum pressures of about 1 kPa to about 5 kPa, about 2.5 kPa to about 7.5 kPa, about 5 kPa to about 10 kPa, about 7.5 kPa to about 12.5 kPa, about 10 kPa to about 15 kPa, about 12.5 kPa to about 17.5 kPa, about 15 kPa to about 20 kPa, about 17.5 kPa to about 22.5 kPa, about 20 kPa to about 25 kPa, about 22.5 kPa to about 27.5 kPa, about 25 kPa to about 30 kPa, about 27.5 kPa to about 32.5 kPa, about 30 kPa to about 35 kPa, about 32.5 kPa to about 37.5 kPa, about 35 kPa to about 40 kPa, or about 40 kPa or greater.

The vacuum pressures that the nonwoven material can withstand without collapsing may depend on a variety of factors. For example, increasing the density, thickness, and weight basis of the nonwoven material; orienting at least some of the fibers vertically; or increasing the yield strength or Young's modulus (e.g., elastic modulus) of the material that forms the fibers generally increases the vacuum pressure that may be applied to the chamber 114. The vacuum pressures that the nonwoven material can withstand without collapsing also depends of the type of nonwoven material (e.g., carded web, needle punched web, etc.). However, increasing the vacuum pressure that may be applied to the chamber 114 without collapsing the nonwoven material may adversely affect some of the other properties of the nonwoven material, such as the flow rate of bodily fluids through the nonwoven material. As such, in some embodiments, selecting the nonwoven material to resist certain vacuum pressures without collapsing may requiring balancing several factors which may prevent the nonwoven material from being able to resist all possible vacuum pressures. In such embodiments, the nonwoven material may be rated to be used with vacuum pressures that are under a certain value or only with certain vacuum sources to prevent the nonwoven material from collapsing. The rating of the nonwoven material may be printed on the fluid collection assembly 100 (e.g., printed on the fluid impermeable barrier 108) or on the packaging that contains the fluid collection assembly 100 to prevent the collapse of the nonwoven material since the rating of the nonwoven material may not be apparent and preventing the collapse of the nonwoven material may be critical to the function of the fluid collection assembly 100.

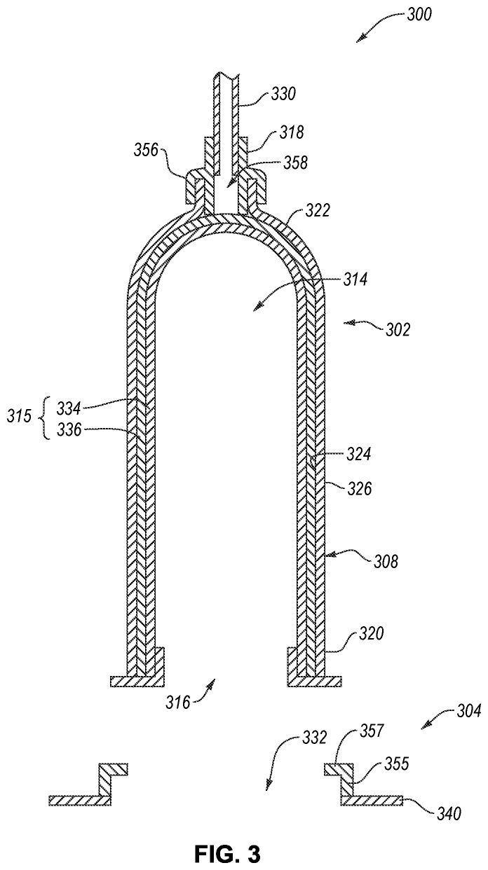

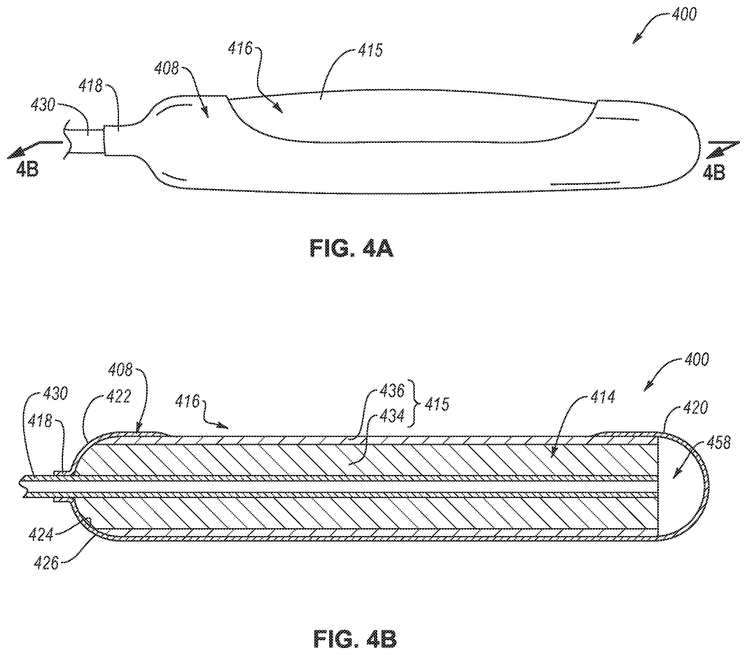

The vacuum pressure that the nonwoven material is configured to resist without collapsing may also be selected based on the fluid impermeable barrier 108. For example, the fluid impermeable barrier 108 of the fluid collection assembly 100 is formed from films which makes the fluid impermeable barrier 108 susceptible to collapsing if not for the nonwoven material. In such an example, the nonwoven material of the porous material 115 may be selected to withstand a relatively high vacuum pressure. However, in an example, the fluid impermeable barrier may be configured to resist collapsing, such as when the fluid impermeable barrier 108 includes a thick silicone or neoprene material, as shown in FIGS. 3-4B. In such an example, the nonwoven material may be configured to withstand a relatively small vacuum pressure (e.g., a vacuum pressure that is smaller than the relatively high vacuum pressure discussed above) without collapsing since the nonwoven material does not have to resist the vacuum pressure by itself.

The nonwoven material of the porous material 115 may be selected to exhibit a density of about 5 kg/m³ to about 10 kg/m³, about 7.5 kg/m³ to about 12.5 kg/m³, about 10 kg/m³ to about 15 kg/m³, about 12.5 kg/m³ to about 17.5 kg/m³, about 15 kg/m³ to about 20 kg/m³, about 17.5 kg/m³ to about 22.5 kg/m³, about 20 kg/m³ to about 25 kg/m³, about 22.5 kg/m³ to about 27.5 kg/m³, about 25 kg/m³ to about 30 kg/m³, about 27.5 kg/m³ to about 32.5 kg/m³, about 30 kg/m³ to about 35 kg/m³, about 32.5 kg/m³ to about 37.5 kg/m³, about 35 kg/m³ to about 37.5 kg/m³, about 35 kg/m³ to about 40 kg/m³, about 37.5 kg/m³ to about 42.5 kg/m³, about 40 kg/m³ to about 45 kg/m³, about 42.5 kg/m³ to about 47.5 kg/m³, or about 45 kg/m³ to about 50 kg/m³. Generally, increasing the density of the nonwoven material increases the strength of the nonwoven material which, in turn, increases the ability of the nonwoven material to resist collapse when the vacuum pressure is applied to the chamber 114. However, increasing the density of the nonwoven material may decrease the porosity of the nonwoven material which decreases the quantity of bodily fluids that may be temporarily stored in the porous material 115 and decrease the flow rate of the bodily fluids through the nonwoven material. As such, the density of the nonwoven material may be selected based on balancing the desired strength, porosity, and flow rate of the bodily fluids through the nonwoven material. For example, the density may be increased when the at least one of the fluid impermeable barrier 108 is unable to resist collapse when the vacuum pressure is applied to the chamber 114, the volume of the chamber 114 is relatively large, or the distance that the bodily fluids needs to travel to the fluid outlet 118 is relatively small.

The nonwoven material of the porous material 115 may be selected to exhibit a thickness that is greater than about 1 mm, such as in ranges of 1 mm to about 3 mm, about 2 mm to about 4 mm, about 3 mm to about 5 mm, about 4 mm to about 6 mm, about 5 mm to about 7 mm, about 6 mm to about 8 mm, about 7 mm to about 9 mm, about 8 mm to about 10 mm, about 9 mm to about 11 mm, about 10 mm to about 12 mm, about 11 mm to about 13 mm, about 12 mm to about 14 mm, about 13 mm to about 15 mm, about 14 mm to about 16 mm, about 15 mm to about 18 mm, about 17 mm to about 20 mm, about 19 mm to about 22 mm, about 21 mm to about 25 mm, or about 24 mm to about 30 mm Increasing the thickness of the nonwoven material generally increases the ability of the nonwoven material to resist complete collapse when the vacuum pressure is applied thereto, increases the volume of bodily fluids that may be temporarily stored in the nonwoven material, and allows greater flexibility in selecting the density and basis weight of the nonwoven material. However, the thickness of the nonwoven material may be limited by the size and functionality of the fluid collection assembly 100. For example, the thickness of the nonwoven material must be selected such that the nonwoven material may be disposed in the chamber 114, along with any other items that may also be disposed in the chamber 114, such as at least one of one or more additional layers of the porous material 115, a conduit 130, or a penis. Further, increasing the thickness of the nonwoven material may make removing substantially all of the bodily fluids from the chamber 114 difficult since the increased thickness dilutes the vacuum pressure in the porous material 115.

The nonwoven material of the porous material 115 may be selected to exhibit a basis weight of about 50 g/m² to about 100 g/m², about 75 g/m² to about 125 g/m², about 100 g/m² to about 150 g/m², about 125 g/m² to about 175 g/m², about 150 g/m² to about 200 g/m², about 175 g/m² to about 225 g/m², about 200 g/m² to about 250 g/m², about 225 g/m² to about 275 g/m², about 250 g/m² to about 300 g/m², about 275 g/m² to about 325 g/m², about 300 g/m² to about 375 g/m², about 350 g/m² to about 450 g/m², or about 400 g/m² to about 500 g/m². The basis weight of the nonwoven material is a function of the density and thickness of the nonwoven material. As such, the basis weight of the nonwoven material may be selected for any of the same reasons as the density and thickness of the nonwoven material.

As previously discussed, the nonwoven material is formed from a plurality of fibers. The plurality of fibers may exhibit an average length and an average lateral dimension (e.g., diameter). In an example, the plurality of fibers may be selected to exhibit an average length that is about 500 μm to about 2 mm, about 1 mm to about 3 mm, about 2 mm to about 4 mm, about 3 mm to about 5 mm, about 4 mm to about 6 mm, about 5 mm to about 7 mm, about 6 mm to about 8 mm, about 7 mm to about 9 mm, about 8 mm to about 1 cm, about 9 mm to about 1.2 cm, about 1 cm to about 1.4 cm, about 1.2 cm to about 1.6 cm, about 1.4 cm to about 1.8 cm, about 1.6 cm to about 2 cm, about 1.8 cm to about 2.25 cm, about 2 cm to about 2.5 cm, about 2.25 cm to about 2.75 cm, about 2.5 cm to about 3 cm, about 2.75 cm to about 3.25 cm, about 3 cm to about 3.5 cm, about 3.25 cm to about 3.75 cm, about 3.5 cm to about 4 cm, about 3.75 cm to about 4.25 cm, about 4 cm to about 4.5 cm, about 4.25 cm to about 4.75 cm, about 4.5 cm to about 5 cm, about 4.75 cm to about 5.5 cm, about 5 cm to about 6 cm, about 5.5 cm to about 6.5 cm, about 6 cm to about 7 cm, about 6.5 cm to about 7.5 cm, about 7 cm to about 8 cm, about 7.5 cm to about 8.5 cm, about 8 cm to about 9 cm, about 8.5 cm to about 9.5 cm, or about 9 cm to about 10 cm. In an example, the fibers may exhibit an average lateral dimension that is about 1 μm to about 2 μm, about 1.5 μm to about 3 μm, about 2 μm to about 4 μm, about 3 μm to about 5 μm, about 4 μm to about 7 μm, about 6 μm to about 10 μm, about 8 μm to about 12.5 μm, about 10 μm to about 15 μm, about 12.5 μm to about 17.5 μm, about 15 μm to about 20 μm, about 17.5 μm to about 25 μm, about 20 μm to about 30 μm, about 25 μm to about 35 μm, about 30 μm to about 40 μm, about 35 μm to about 45 μm, about 40 μm to about 50 μm, about 45 μm to about 55 μm, about 50 μm to about 60 μm, about 55 μm to about 65 μm, about 60 μm to about 70 μm, about 65 μm to about 75 μm, about 70 μm to about 80 μm, about 75 μm to about 85 μm, about 80 μm to about 90 μm, about 85 μm to about 95 μm, or about 90 μm to about 100 μm. The average length and average lateral dimension of the fibers may be selected such that the fibers exhibits an average aspect ratio. For example, the average length and average lateral dimension of the fibers may be selected such that the fibers exhibit an average aspect ratio (average length:average lateral dimension) of about 100:1 to about 200:1, about 150:1 to about 250:1, about 200:1 to about 300:1, about 250:1 to about 350:1, about 300:1 to about 400:1, about 350:1 to about 450:1, about 400:1 to about 500:1, about 450:1 to about 550:1, about 500:1 to about 600:1, about 550:1 to about 650:1, about 600:1 to about 700:1, about 650:1 to about 750:1, about 700:1 to about 800:1, about 750:1 to about 850:1, about 800:1 to about 900:1, about 850:1 to about 950:1, or about 900:1 to about 1,000:1.

The average length, average lateral dimension, and the average aspect ratio of the fibers may be selected based on a number of factors. In an example, increasing the aspect ratio (e.g., decreasing the average length and/or increasing the average lateral dimension) increases the durability of the nonwoven material but may decrease the strength of the nonwoven material. In an example, increasing the aspect ratio (e.g., increasing average length) of the fibers may increase the mechanical binding of the fibers. For instance, increasing the aspect ratio of the fibers facilitates entanglement of the fibers which increases the strength and durability of the nonwoven material. The entanglement of the fibers may also preclude or minimize the amount of other binding techniques that are applied to the nonwoven material, such as heat, chemical binding, or other mechanical binding (e.g., further entanglement caused by needle punching or high pressure water jets). However, increasing the aspect ratio of the fibers may make dispersion of the fibers more difficult (e.g., uniformity of the nonwoven material is decreased or more difficult to achieve). Further, increasing the aspect ratio may limit the type of nonwoven material that may include the fibers. For instance, fibers with large average lengths (e.g., large aspect ratios) may not be used in carded webs and may have to be used in air laid webs. In an example, decreasing the aspect ratio may decrease the entanglement of the fibers thereby necessitating further binding of the fibers. As such, the average length, average lateral dimension, and average aspect ratio of the fibers may be selected based on the desired strength, mechanical binding between the fibers, the amount of processing of the nonwoven material (e.g., is further processing to increasing the binding via heat, etc. desired), the type of nonwoven material that includes the fibers, the uniformity of the fibers, etc.

Generally, the fibers of the nonwoven material are formed from a hydrophilic material. The hydrophilicity of the fibers pulls the bodily fluids into the nonwoven material thereby maintaining the skin of the region of the patient dry. The hydrophilicity also pulls the bodily fluids towards the fluid outlet 118 in conjunction with the orientation of the fibers, gravity, and the vacuum pressure. For example, the fibers of the nonwoven material may exhibit a contact angle with water (e.g., a major components of most bodily fluids) of about 0° to about 15°, about 10° to about 25°, about 20° to about 35°, about 30° to about 45°, about 50° to about 65°, about 60° to about 75°, or about 70° to about 90°. Generally, increasing the contact angle increases the rate at which the bodily fluids are pulled into the nonwoven material but may make removing the bodily fluids from the nonwoven more difficult. In an embodiment, the fibers of the nonwoven material may be formed from a hydrophobic material coated with a hydrophilic material or otherwise treated such that the fibers exhibit any of the contact angles disclosed above.

In an example, the fibers of the nonwoven material may be formed from synthetic fibers, such as polyester, polypropylene, polyurethane, polyolefin, polycarbonate, polyvinyl chloride, polyacrylic, nylon, other synthetic fibers, or combinations thereof. In an example, the fibers of the nonwoven material may be formed from natural fibers, such as low grade cotton waste. The natural fibers may be cheaper than the synthetic fibers and may be biodegradable. In some instances, the natural fibers may also exhibit better bonding and absorption properties than some synthetic fibers. However, the natural fibers may exhibit a durability that is less than some synthetic fibers. In an example, the fibers may be formed from synthetic and natural fibers. In an example, the fibers of the nonwoven material may be formed from hollow fibers which may reduce the density and basis weight of the nonwoven material with minimal effect on the strength or durability of the nonwoven material.

Generally, the average person discharges urine at a rate of about 6 ml/s to about 50 ml/s, such as at a rate of about 10 ml/s to about 25 ml/s. The rate at which the person urinate may vary, such as based on the size of the person and the age of the person. The nonwoven material may be selected to exhibit a flow rate that is comparable to the rate at which the fluid collection assembly 100 receives the bodily fluids to prevent oversaturation of the nonwoven material with bodily fluids which may cause leaks. For example, the nonwoven material may be selected to exhibit a flow rate that is greater than about 6 ml/s, greater than about 10 ml/s, greater than about 20 ml/s, greater than about 30 ml/s, greater than about 40 ml/s, greater than about 50 ml/s, or in ranges of about 6 ml/s to about 10 ml/s, about 8 ml/s to about 12 ml/s, about 10 ml/s to about 15 ml/s, about 12.5 ml/s to about 17.5 ml/s, about 15 ml/s to about 20 ml/s, about 17.5 ml/s to about 22.5 ml/s, about 20 ml/s to about 25 ml/s, about 22.5 ml/s to about 27.5 ml/s, about 25 ml/s to about 30 ml/s, about 27.5 ml/s to about 35 ml/s, about 30 ml/s to about 40 ml/s, about 35 ml/s to about 45 ml/s, or about 40 ml/s to about 50 ml/s. As used herein, the flow rate may refer to the flow rate of the bodily fluids in the nonwoven material when the nonwoven material is at least one of saturated with the bodily fluids, not saturated with the bodily fluids, any of the vacuum pressures disclosed herein are applied to the chamber 114, or when no vacuum pressure is applied to the chamber 114 (e.g., the bodily fluids flow only due to wicking and gravity).

The flow rate of the bodily fluids through the nonwoven material may depend on a number of factors. In an example, the flow rate may depend inversely on the density and weight basis of the nonwoven material, wherein increasing the density and/or weight basis of the nonwoven material may decrease the flow rate of the nonwoven material and vice versa. In an example, the flow rate may depend on the material (e.g., hydrophilicity of the material) that forms the fibers. In an example, the flow rate may increase with increasing thickness since increasing the thickness increases the cross-sectional area through which the bodily fluids may flow. In an example, the flow rate may depend on the type of nonwoven material (e.g., carded web, needle punched web, etc.) since each type of nonwoven material may exhibit different flow rates and absorptions rates when all other factors are the same, as discussed above.

The fluid collection assembly 100 may be rated based on the fluid flow rate of the nonwoven material. For example, when the flow rate of the nonwoven is less than the flow rate of urine for the average person (e.g., less than 50 ml/s or less than 25 ml/s), the fluid collection assembly 100 may indicate that the fluid collection assembly 100 may only be used with certain individuals to decrease the likelihood that the fluid collection assembly 100 leaks. The rating may be provided on the fluid collection assembly 100 itself or on packaging that at least initially included the fluid collection assembly 100. Providing the rating, in some embodiments, may be critical to the function of the fluid collection assembly 100 to prevent the fluid collection assembly 100 from leaking.

The nonwoven material of the porous material 115 may be selected to be able to remove the bodily fluids therein within a certain time period (“evacuation time”). For example, the average person urinates during a period of about 8 seconds to about 36 seconds. The nonwoven material may be selected to exhibit an evacuation time that is comparable to the average urination to prevent the bodily fluids from leaking therefrom. For example, the nonwoven material may be selected to have an evacuation time that is at most 6 seconds, at most about 10 seconds, at most about 15 seconds, at most about 20 second, at most about 25 seconds, at most about 30 seconds, at most about 35 seconds, or in ranges of about 6 seconds to about 15 seconds, about 10 seconds to about 20 seconds, about 15 seconds to about 25 seconds, about 20 seconds to about 30 seconds, or about 25 seconds to about 35 seconds. As used herein, the evacuation time refers to the time to remove substantially all (e.g., at least about 50%, at least about 75%, or at least about 90%) of the bodily fluids from the nonwoven material when the nonwoven material is saturated or nearly saturated with the bodily fluids when any of the vacuum pressures disclosed herein are applied to the chamber 114. The evacuation time may depend on the fluid flow rate of the bodily fluids in the nonwoven material and the volume (e.g., thickness) of the nonwoven material.

The nonwoven materials disclosed herein may exhibit any combination of the properties disclosed herein. For example, the nonwoven materials disclosed herein may exhibit any of at least two of the types of nonwoven material (e.g., carded web, needle punched web, etc.), vacuum pressures without collapsing, densities, thicknesses, basis weights, average lengths, average lateral dimensions, aspect ratios, hydrophilicities, compositions, flow rate, or evacuation times disclosed above. As such, it is noted that at least some of the nonwoven materials disclosed herein may not exhibit 11 or less (e.g., 10 or less, 8 or less, 6 or less, 5 or less, 4 or less, 3 or less, 2 or less, or 1) of the types of nonwoven material, vacuum pressures without collapsing, densities, thicknesses, basis weights, average lengths, average lateral dimensions, aspect ratios, hydrophilicities, compositions, flow rate, or evacuation times disclosed above.

Referring back to FIGS. 1A and 1B, in an embodiment, the porous material 115 may be a sheet. Forming the porous material 115 as a sheet may facilitate the manufacturing of the fluid collection assembly 100. For example, forming the porous material 115 as a sheet allows the first panel 110, the second panel 112, and the porous material 115 to each be sheets. During the manufacturing of the fluid collection assembly 100, the first panel 110, the second panel 112, and the porous material 115 may be stacked and then attached to each other in the same manufacturing step. For instance, the porous material 115 may exhibit a shape that is the same size or, more preferably, slightly smaller than the size of the first panel 110 and the second panel 112. As such, attaching the first panel 110 and the second panel 112 together along the outer edges thereof may also attach the porous material 115 to the first panel 110 and the second panel 112. The porous material 115 may be slightly smaller than the first panel 110 and the second panel 112 such that the first panel 110 and/or the second panel 112 extend around the porous material 115 such that the porous material 115 does not form a passageway through the fluid impermeable barrier 108 through which the bodily fluids may leak. Also, attaching the porous material 115 to the first panel 110 and/or the second panel 112 may prevent the porous material 115 from significantly moving in the chamber 114, such as preventing the porous material 115 from bunching together near the fluid outlet 118. In an example, the porous material 115 may be attached to the first panel 110 or the second panel 112 (e.g., via an adhesive) before or after attaching the first panel 110 to the second panel 112. In an example, the porous material 115 may merely be disposed in the chamber 114 without attaching the porous material 115 to at least one of the first panel 110 or the second panel 112. In an embodiment, as will be discussed in more detail below, the porous material 115 may exhibit shapes other than a sheet, such as a hollow generally cylindrical shape.

Generally, the sheath 102 is substantially flat when the penis is not in the penis receiving area 128 and the sheath 102 is resting on a flat surface. The sheath 102 is substantially flat because the fluid impermeable barrier 108 is formed from the first panel 110 and the second panel 112 instead of a generally tubular fluid impermeable barrier. Further, as previously discussed, the porous material 115 may be a sheet, which also causes the sheath 102 to be substantially flat. The sheath 102 may also be substantially flat because the fluid collection assembly 100 may not include relatively rigid rings or caps that exhibit a rigidity that is greater than the portions of the fluid impermeable barrier 108 thereabout since such rings and caps may inhibit the sheath 102 being substantially flat. It is noted that the sheath 102 is described as being substantially flat because at least one of the porous material 115 may cause a slight bulge to form in the sheath 102 depending on the thickness of the porous material 115, the fluid outlet 118 and/or conduit 130 may cause a bulge thereabout, or the base 104 may pull on portions of the sheath 102 thereabout. It is also noted that the sheath 102 may also be compliant and, as such, the sheath 102 may not be substantially flat during use since, during use, the sheath 102 may rest on a non-flat surface (e.g., may rest on the testicles, the perineum, and/or between the thighs) and the sheath 102 may conform to the surface of these shapes. It is noted that the sheath 102 is not illustrated as being substantially flat in FIG. 1B to illustrate the penis receiving area 128.

The ability of the sheath 102 to be substantially flat when the penis is not in the penis receiving area 128 and the sheath 102 is resting on a flat surface allows the fluid collection assembly 100 to be used with a buried and a non-buried penis. For example, when the fluid collection assembly 100 is being used with a buried penis, the penis does not extend into the penis receiving area 128 which causes the sheath 102 to lie relatively flat across the aperture 132 of the base 104. When the sheath 102 lies relatively flat across the aperture 132, the porous material 115 extends across the opening 116 and the aperture 132 and is in close proximity to the buried penis. As such, the porous material 115 prevents or inhibits pooling of bodily fluids discharged from the buried penis against the skin of the individual since the porous material 115 will receive and remove at least a significant portion of the bodily fluids that would otherwise pool against the skin of the individual. Thus, the skin of the individual remains dry thereby improving comfort of using the fluid collection assembly 100 and preventing skin degradation. However, unlike other conventional fluid collection assemblies that are configured to be used with buried penises, the fluid collection assembly 100 may still be used with a non-buried penis since the non-buried penis can still be received into the penis receiving area 128, even when the penis is fully erect. Additionally, the ability of the sheath 102 to be substantially flat allows the fluid collection assembly 100 to be used more discretely than if the sheath 102 was not substantially flat thereby avoiding possibly embarrassing scenarios.

When the sheath 102 is substantially flat, the porous material 115 occupies substantially all of the chamber 114 and the penis receiving area 128 is collapsed. In other words, the sheath 102 may not define an region that is constantly unoccupied by the porous material 115. When the porous material 115 occupies substantially all of the chamber 114, the bodily fluids discharged into the chamber 114 are unlikely to pool for significant periods of time since pooling of the bodily fluids may cause sanitation issues, cause an odor, and/or may cause the skin of the individual to remain in contact with the bodily fluids which may cause discomfort and skin degradation.

As previously discussed, the first panel 110, the second panel 112, and the porous material 115 may be selected to be relatively flexible. The first panel 110, the second panel 112, and the porous material 115 are relatively flexible when the first panel 110, the second panel 112, and the porous material 115, respectively, are unable to maintain their shape when unsupported. The flexibility of the first panel 110, the second panel 112, and the porous material 115 may allow the sheath 102 to be substantially flat, as discussed above. The flexibility of the first panel 110, the second panel 112, and the porous material 115 may also allow the sheath 102 to conform to the shape of the penis even when the size and shape of the penis changes (e.g., becomes erect) and to minimize any unoccupied spaces in the chamber 114 in which bodily fluids may pool.

As previously discussed, the fluid collection assembly 100 includes a base 104 that is configured to be attached to the sheath 102. For example, the base 104 is configured to be permanently attached to the sheath 102. The base 104 is configured to be permanently attached to the sheath 102 when, for example, when the fluid collection assembly 100 is provided with the base 104 permanently attached to the sheath 102 or the base 104 is provided without being permanently attached to the sheath 102 but is configured to be permanently attached to the sheath 102 at some point in the future. Permanently attached means that the sheath 102 cannot be detached from the base 104 without damaging at least one of the sheath 102 or the base 104, using a blade to separate the sheath 102 from the base 104, and/or using chemicals to dissolve the adhesive that attaches the sheath 102 from the base 104. The base 104 may be permanently attached to the sheath 102 using an adhesive, sewing, heat sealing, RF welding, or US welding. In an embodiment, the base 104 is configured to be reversibly attached to the sheath 102.

As previously discussed, the base 104 includes an aperture 132. The base 104 is permanently attached to the first end region 120 of the sheath 102 such that the aperture 132 is aligned with the opening 116.

The base 104 is sized, shaped, and made of a material to be coupled to the skin that surrounds the penis (e.g., mons pubis, thighs, testicles, and/or perineum) and have the penis disposed therethrough. For example, the base 104 may define an aperture 132 configured to have the penis positioned therethrough. In an example, the base 104 may exhibit the general shape or contours of the skin surface that the base 104 is configured to be coupled with. The base 104 may be flexible, thereby allowing the base 104 to conform to any shape of the skin surface and mitigate the base 104 pulling the on skin surface. The base 104 may extend laterally past the sheath 102 thereby increasing the surface area of the skin of the individual to which the fluid collection assembly 100 may be attached compared to a substantially similar fluid collection assembly 100 that did not include a base.

The base 104 may be configured to be attached to the skin about the urethral opening of the patient using any suitable technique. For example, the base 104 may include a chemical adhesive (e.g., hydrogel) or a dry adhesive that is configured to attach the base 104 to the skin about the urethral opening.

The fluid collection assembly 100 includes a conduit 130. The conduit 130 may be the same or substantially similar to any of the assembly tubes disclosed herein. An inlet of the conduit 130 may be located at or near the second end region 122 of the sheath 102 which is expected to be the gravimetrically low point of the chamber 114 when worn by a user. Locating the inlet of the conduit 130 at or near the second end region 122 of the sheath 102 enables the conduit 130 to receive more of the bodily fluids than if the inlet of the conduit 130 was located elsewhere and reduce the likelihood of pooling (e.g., pooling of the bodily fluids may cause microbe growth and foul odors). For instance, the bodily fluids in porous material 115 flow into the porous material 115 due to capillary forces. However, the bodily fluids may exhibit a preference to flow in the direction of gravity, especially when at least a portion of the porous material 115 is saturated with the bodily fluids. Accordingly, the inlet of the conduit 130 may be located in the fluid collection assembly 100 in a position expected to be the gravimetrically low point in the fluid collection assembly 100 when worn by a user.

In an example, the conduit 130 is configured to be at least insertable into the chamber 114, such as into the penis receiving area 128. In such an example, the conduit 130 may include one or more markers (not shown) on an exterior thereof that are located to facilitate insertion of the conduit 130 into the chamber 114. For example, the conduit 130 may include one or more markings thereon that are configured to prevent over or under insertion of the conduit 130. In another example, the conduit 130 may include one or more markings thereon that are configured to facilitate correct rotation of the conduit 130 relative to the chamber 114. The one or more markings may include a line, a dot, a sticker, or any other suitable marking.

The conduit 130 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some examples, the conduit 130 may include silicon or latex. In some examples, the conduit 130 may include one or more portions that are resilient, such as to by having one or more of a diameter or wall thickness that allows the conduit 130 to be flexible.

As described in more detail below, the conduit 130 is configured to be coupled to, and at least partially extend between, one or more of the fluid storage container (not shown) and the vacuum source (not shown). In some examples, the vacuum source may be remotely located from the fluid collection assembly 100. In such examples, the conduit 130 may be fluidly connected to the fluid storage container, which may be disposed between the vacuum source and the fluid collection assembly 100.

During operation, a male using the fluid collection assembly 100 may discharge bodily fluids (e.g., urine) into the chamber 114. The bodily fluids may pool or otherwise be collected in the chamber 114. At least some of the bodily fluids may be pulled through the interior of the conduit 130 via the inlet. The fluid may be drawn out of the fluid collection assembly 100 via the vacuum/suction provided by the vacuum source.

Further examples of male fluid collection assemblies are disclosed in U.S. Provisional Patent Application No. 63/067,542 filed on Aug. 19, 2020, the disclosure of which is incorporated herein, in its entirety, by this reference.

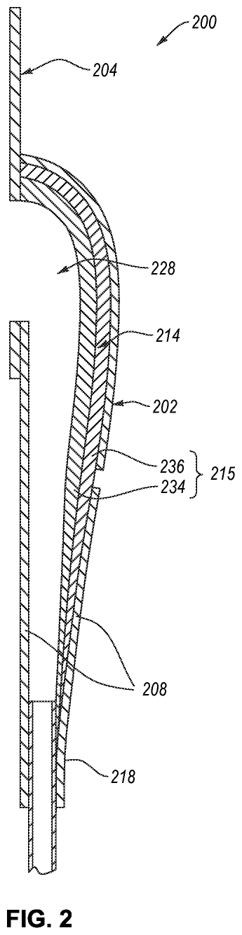

FIG. 2 is a cross-sectional schematic of a fluid collection assembly 200 according to an embodiment. Except as otherwise disclosed herein, the fluid collection assembly 200 is the same or substantially similar to any of the fluid collection assemblies disclosed herein. For example, similar to the fluid collection assembly 100 illustrated in FIGS. 1A and 1B, the fluid collection assembly 200 may include a sheath 202 and a base 204. The sheath 202 may include a fluid impermeable barrier 208 defining a chamber 214 and at least one porous material 215 disposed in the chamber 214.

Unlike the porous material 115 illustrated in FIG. 1B, the porous material 215 includes a plurality of layers. For example, the porous material 215 will be discussed as including a first layer 234 and a second layer 236. The first layer 234 may at least partially define the penis receiving area 228, while at least a portion of the second layer 236 may be spaced from the penis receiving area 228 by at least a portion of the first layer 234. It is noted that the porous material 215 may include three or more layers, without limitation. The same principles discussed with regard to the first and second layers 234, 236 may be applied when the porous material 215 includes three or more layers.

In an embodiment, each of the plurality of layers of the porous material 215 may be formed from nonwoven materials, such as different nonwoven materials. In such an embodiment, the first layer 234 may be formed from a first nonwoven material and the second layer 236 may be formed from a second nonwoven material. The first nonwoven material may exhibit one or more properties that are different than the second nonwoven material. In an example, the first nonwoven material may wick one or more bodily fluids vertically better than the second nonwoven material which may better remove the bodily fluids from being in contact with the patient. The second nonwoven material may wick the one or more bodily fluids horizontally better than the first nonwoven material thereby moving the bodily fluids towards the fluid outlet 218. For instance, the first nonwoven material may include a vertically lapped nonwoven fabric and the second nonwoven material may include a carded web, a horizontally lapped nonwoven fabric, or a crossed lapped nonwoven fabric. In an example, the first nonwoven material may exhibit better wicking properties (e.g., fluid flow rates) than the second nonwoven material and the second nonwoven material may better resist collapse due to the vacuum pressure than the first nonwoven material, or vice versa.

In an embodiment, at least one of the plurality of layers of the porous material 215 may be formed from nonwoven material(s) and at least one remaining layer of the porous material 215 may be formed from a woven material. The porous material 215 may include a woven material for a variety of reasons. In an example, some of the nonwoven materials disclosed herein may be uncomfortable when directly contacting the skin (e.g., penis) of the patient. The nonwoven material may be uncomfortable because, for example, the nonwoven materials may exhibit at least one of a rough surface texture, include fibers protruding therefrom, or be formed from stiff fibers. As such, the first layer 234 may be formed from a woven material and the second layer 236 may be formed from a nonwoven material which allows the woven material to interface with the skin of the patient instead of the nonwoven material. In an example, the first layer 234 may include a hydrophobic woven material and the second layer 236 may include a hydrophilic nonwoven material. The bodily fluids may flow through the hydrophobic woven because of the vacuum pressure. However, the hydrophobic woven material may inhibit bodily fluids from flowing back through the hydrophobic woven material. In other words, the hydrophobic woven material may keep the patient dry.

The porous material 215 may include any suitable woven material. In an example, the woven material may be formed from a woven fabric, such as a gauze (e.g., a silk, linen, or cotton gauze), another soft fabric, or another smooth fabric. In an example, the woven material may include a polymer, such as nylon, polyester, polyurethane, polyethylene, polypropylene rayon, acrylic, nomex, Kevlar, Teflon, etc. In a particular example, the woven material may include spun nylon fibers. In an example, the woven material may include natural fibers, such as cotton, wool, silk, wood pulp, or combinations thereof. In an example, the woven material may include carbon, metal fibers, or ceramic fibers. In an example, the woven material may be selected to be exhibit substantially no absorption after the woven material is exposed to the bodily fluids and removed from the bodily fluids for a time. As used herein, “substantially no absorption” may allow for nominal amounts of absorption of the bodily fluids into the woven material, such as less than about 10 wt % of the dry weight of the wicking material, less than about 7 wt %, less than about 5 wt %, less than about 3 wt %, less than about 2 wt %, less than about 1 wt %, or less than about 0.5 wt % of the dry weight of the woven material. The woven material may also wick the bodily fluids generally towards an interior of the chamber 114. In an embodiment, the woven material may include at least one absorbent or adsorbent material. IN an example, the woven material may include a combinations of any of the materials disclosed herein.

The porous materials disclosed herein may be used with other fluid collection assemblies other than the fluid collection assemblies illustrated in FIG. 1A-2B. For example, FIG. 3 is a cross-sectional schematic of a fluid collection assembly 300, according to an embodiment. The fluid collection assembly 300 is a male fluid collection assembly configured to receive one or more bodily fluids from a male urethral opening. Except as otherwise disclosed herein, the fluid collection assembly 300 is the same or substantially similar to any of the fluid collection assemblies disclosed herein.

The fluid collection assembly 300 includes a sheath 302 and a base 304 and a sheath 302. The sheath 302 includes (e.g., may be formed from) a fluid impermeable barrier 308 that is sized and shaped to fit into the hollowed region of the base 304. For example, the sheath 302 may be generally tubular or cup-shaped, as shown. The generally tubular or cup-shaped fluid impermeable barrier 308 may at least partially define the outer surface 326 of the sheath 302. The fluid impermeable barrier 308 may be similar or identical to the fluid impermeable barrier 108 as disclosed herein, in one or more aspects. For example, the fluid impermeable barrier 308 may be constructed of any of the materials disclosed herein for the fluid impermeable barrier 108. The fluid impermeable barrier 308 at least partially defines the chamber 314. For example, the inner surface 324 of the fluid impermeable barrier 308 at least partially defines the perimeter of the chamber 314. The chamber 314 may be similar or identical to the chamber 114 in one or more aspects. For example, the chamber 314 may at least temporarily retain fluids therein. As shown, the fluid collection assembly 300 is configured to receive a penis and may include the porous material 315 therein.

The fluid collection assembly 300 includes at least one porous material 315. The porous material 315 may be similar or identical to any of the porous materials disclosed herein in one or more aspects. The porous material 315 includes at least one nonwoven material, such as any of the nonwoven material disclosed herein. In an example, as illustrated, the porous material 315 includes a plurality of layers (e.g., at least a first layer 334 and a second layer 336) similar to the porous material 215 illustrated in FIG. 2. At least one of the plurality of layers of the porous material 315 includes a nonwoven material and, optionally, a remainder of the layers of the porous material 315 includes one or more woven materials. In an example, the porous material 315 may include a single layer of nonwoven material similar to the porous material 115 illustrated in FIG. 1B. In an embodiment, the porous material 315 may exhibit a generally hollow cylindrical shape. In such an embodiment, the vertical direction of the porous material 315 is generally parallel to a radius of the cylinder and the horizontal direction is in-plane with the length and circumference of the cylinder.

The sheath 302 also includes at least a portion of the conduit 330 therein, such as at least partially disposed in the chamber 314. For example, the conduit 330 may extend from the sheath 302 at the second end region 322 at least partially towards a first end region 320 at least proximate to the aperture 332. The first end region 320 may be disposed near or on the skin around the male urethra (e.g., on the penis or pubic area therearound).

In some examples, the fluid impermeable barrier 308 may be constructed of a material and/or have a thickness that allows the sheath 302 to collapse when placed under vacuum, such as to remove air around a penis in the fluid collection assembly 300 during use. In such examples, the conduit 330 may extend only to or into the second end region 322 in the chamber 314 (e.g., not through to the area adjacent the opening 316). In such examples, urine may be collected and removed from the fluid collection assembly 300 at the first end region 320. It is noted that the porous material 315 may not collapse when the sheath 302 collapses thereby allowing bodily fluids to flow through the fluid collection assembly 300.

In an example, portions of the chamber 314 may be substantially empty due to the varying sizes and rigidity of the male penis. However, in some examples, the outermost regions of the chamber 314 (e.g., periphery of the interior regions of the sheath 302) may include porous material 315. For example, the porous material 315 may be bonded to the inner surface 324 of the fluid impermeable barrier 308. The porous material 315 may be positioned (e.g., at the distal end of the chamber 314) to blunt a stream of urine from the male urethra thereby limiting splashing and/or to direct the bodily fluids to a selected region of the chamber 314. Since the chamber 314 is substantially empty (e.g., substantially all of the chamber 314 forms a reservoir), the fluids are likely to pool at a gravimetrically low point of the chamber 314. The gravimetrically low point of the chamber 314 may be at an intersection of the skin of an patient and the fluid collection assembly 300, a corner formed in the sheath 302, or another suitable location depending on the orientation of the patient.

The base 304 is sized, shaped, and made of a material to be coupled to skin that surrounds the male urethra and have the male urethra positioned therethrough. For example, the base 304 define an aperture 332 in the base 304. The base 304 is sized and shaped to be positioned around the male urethra (e.g., positioned around and/or over the penis) and the aperture 332 may be configured to have the male urethra positioned therethrough. The base 304 may also be sized, shaped, made of a material, or otherwise configured to be coupled (e.g., adhesively attached, such as with a hydrogel adhesive) to the skin around the male urethra (e.g., around the penis). In an example, the base 304 may exhibit the general shape or contours of the skin surface that the base 304 is selected to be coupled with. The base 304 may be flexible thereby allowing the base 304 to conform to any shape of the skin surface. The base 304 may include a longitudinally extending flange 355 and a laterally extending flange 357 extending inwardly from the longitudinal extending flange 355. The longitudinally extending flange 355 and the laterally extending flange 357 define a hollowed region that is configured to receive (e.g., seal against) the sheath 302. The base 304 may include an adhesive, such as a chemical or dry adhesive, disposed on the base 304 which is configured to attach the base 304 to the region about the urethral opening.

In some examples, the fluid collection assembly 300 includes a cap 356 at a second end region 322. The cap 356 defines an interior channel through which the fluids may be removed from the fluid collection assembly 300. The interior channel is in fluid communication with the chamber 314. The cap 356 may be disposed over at least a portion of the second end region 322 of one or more of the fluid impermeable barrier 308 or the porous material 315. The cap 356 may be made of a polymer, rubber, or any other fluid impermeable material. The cap 356 may be attached to one or more of the fluid impermeable barrier 308, the porous material 315, or the conduit 330. The cap 356 may cover at least a portion of the second end region 322 of the fluid collection assembly 300. The cap 356 may laterally extend a distance from the sheath 302. The cap 356 defines a fluid outlet 318 that is sized and configured to receive and fluidly seal against the conduit 330, such as within the interior channel. The conduit 330 may extend a distance within or through the cap 356, such as to the porous material 315, through the porous material 315, or to a point set-off from the porous material 315. In the latter example, the interior channel of the cap 356 may define a reservoir 358 therein. In some examples (not shown), the cap 356 may be omitted.

The reservoir 358 is an unoccupied portion of fluid collection assembly 300 such as in the cap 356 and is void of other material. In some examples, the reservoir 358 is defined at least partially by the porous material 315 and the cap 356. During use, the fluids that are in the chamber 314 may flow through the porous material 315 to the reservoir 358. The reservoir 358 may store at least some of the fluids therein and/or position the fluids for removal by the conduit 330. In some examples, at least a portion of the porous material 315 may extend continuously between at least a portion of the opening of the interior channel and chamber 314 to wick any fluid from the opening directly to the reservoir 358.

The base 304, the sheath 302, the cap 356, and the conduit 330 may be attached together using any suitable method. For example, at least two of the base 304, the sheath 302, the cap 356, or the conduit 330 may be attached together using at least one of an interference fit, an adhesive, stitching, welding (e.g., ultrasonic welding), tape, any other suitable method, or combinations thereof.

In some examples (not shown), the fluid collection assembly 300 may have a one piece design, with one or more of the sheath 302, the base 304, and the cap 356 being a single, integrally formed piece.

Further examples of male fluid collection assemblies that may be used here are disclosed in U.S. patent application Ser. No. 16/433,773 filed on Jun. 6, 2019, the disclosure of which is incorporated herein, in its entirety, by this reference.

The porous materials disclosed herein that include at least one nonwoven material may be used with a female fluid collection assembly. FIG. 4A is an isometric view of a fluid collection assembly 400, according to an embodiment. FIG. 4B is a cross-sectional schematic of the fluid collection assembly 400 taken along plane 4B-4B shown in FIG. 4A, according to an embodiment. The fluid collection assembly 400 is a female fluid collection assembly that is configured to be disposed adjacent to a female urethral opening. Except as otherwise disclosed herein, the fluid collection assembly 400 is the same or substantially similar to any of the fluid collection assemblies disclosed herein. The fluid collection assembly 400 includes a fluid impermeable barrier 408, at least one porous material 415 disposed in a chamber 414 defined by the fluid impermeable barrier 408, and an optional conduit 430 at least partially disposed within the chamber 414.

The fluid impermeable barrier 408 at least partially defines a chamber 414 (e.g., interior region) and an opening 416. For example, the interior surface(s) 424 of the fluid impermeable barrier 408 at least partially defines the chamber 414 within the fluid collection assembly 400. The fluid impermeable barrier 408 temporarily stores the bodily fluids in the chamber 414. The fluid impermeable barrier 408 may be formed of any suitable fluid impermeable material(s), such as any of the fluid impermeable materials disclosed herein. As such, the fluid impermeable barrier 408 substantially prevents the bodily fluids from passing through the fluid impermeable barrier 408. In an example, the fluid impermeable barrier 408 may be air permeable and fluid impermeable. In such an example, the fluid impermeable barrier 408 may be formed of a hydrophobic material that defines a plurality of pores. At least one or more portions of at least an outer surface 426 of the fluid impermeable barrier 408 may be formed from a soft and/or smooth material, thereby reducing chaffing.

In some examples, the fluid impermeable barrier 408 may be tubular (ignoring the opening 416), such as substantially cylindrical (as shown), oblong, prismatic, or flattened tubes. During use, the outer surface 426 of the fluid impermeable barrier 408 may contact the patient. The fluid impermeable barrier 408 may be sized and shaped to fit in the gluteal cleft between the legs of a female user.

The opening 416 provides an ingress route for fluids to enter the chamber 414. The opening 416 may be defined by the fluid impermeable barrier 408 such as by an inner edge of the fluid impermeable barrier 408. For example, the opening 416 is formed in and extends through the fluid impermeable barrier 408, from the outer surface 426 to the inner surface 424, thereby enabling bodily fluids to enter the chamber 414 from outside of the fluid collection assembly 400. The opening 416 may be an elongated hole in the fluid impermeable barrier 408. For example, the opening 416 may be defined as a cut-out in the fluid impermeable barrier 408. The opening 416 may be located and shaped to be positioned adjacent to a female urethra.

The fluid collection assembly 400 may be positioned proximate to the female urethral opening and the bodily fluids may enter the chamber 414 of the fluid collection assembly 400 via the opening 416. The fluid collection assembly 400 is configured to receive the bodily fluids into the chamber 414 via the opening 416. When in use, the opening 416 may have an elongated shape that extends from a first location below the urethral opening (e.g., at or near the anus or the vaginal opening) to a second location above the urethral opening (e.g., at or near the top of the vaginal opening or the pubic hair).

The opening 416 may have an elongated shape because the space between the legs of a female is relatively small when the legs of the female are closed, thereby only permitting the flow of the bodily fluids along a path that corresponds to the elongated shape of the opening 416 (e.g., longitudinally extending opening). The opening 416 in the fluid impermeable barrier 408 may exhibit a length that is measured along the longitudinal axis of the fluid collection assembly 400 that may be at least about 20% of the length of the fluid collection assembly 400, such as about 25% to about 50%, about 40% to about 60%, about 50% to about 75%, about 65% to about 85%, or about 75% to about 95% of the length of the fluid collection assembly 400.

The opening 416 in the fluid impermeable barrier 408 may exhibit a width that is measured transverse to the longitudinal axis of the fluid collection assembly 400 that may be at least about 40% of the circumference of the fluid collection assembly 400, such as about 25% to about 50%, about 40% to about 60%, about 50% to about 75%, about 65% to about 85%, or about 75% to about 400% of the circumference of the fluid collection assembly 400. The opening 416 may exhibit a width that is greater than 50% of the circumference of the fluid collection assembly 400 since the vacuum (e.g., suction) through the conduit 430 pulls the fluid through the porous material 415 and into the conduit 430. In some examples, the opening 416 may be vertically oriented (e.g., having a major axis parallel to the longitudinal axis of the fluid collection assembly 400). In some examples (not shown), the opening 416 may be horizontally oriented (e.g., having a major axis perpendicular to the longitudinal axis of the fluid collection assembly 400). It is noted that the orientations of the opening 416 may be different than the orientations of the fibers of the nonwoven material and the direction of flow in the porous material 415. In an example, the fluid impermeable barrier 408 may be configured to be attached to the patient, such as adhesively attached (e.g., with a hydrogel adhesive) to the patient. According to an example, a suitable adhesive is a hydrogel layer.

In some examples, the fluid impermeable barrier 408 may define an fluid outlet 418 sized to receive the conduit 430. The at least one conduit 430 may be disposed in the chamber 414 via the fluid outlet 418. The fluid outlet 418 may be sized and shaped to form an at least substantially fluid tight seal against the conduit 430 or the at least one tube thereby substantially preventing the bodily fluids from escaping the chamber 414.

The fluid impermeable barrier 408 may include markings thereon, such as one or more markings to aid a user in aligning the fluid collection assembly 400 on the patient. For example, a line on the fluid impermeable barrier 408 (e.g., opposite the opening 416) may allow a healthcare professional to align the opening 416 over the urethra of the patient. In examples, the markings may include one or more of alignment guide or an orientation indicator, such as a stripe or hashes. Such markings may be positioned to align the fluid collection assembly 400 to one or more anatomical features such as a pubic bone, etc.

The fluid collection assembly 400 includes porous material 415 disposed in the chamber 414. The porous material 415 may cover at least a portion (e.g., all) of the opening 416. The porous material 415 is exposed to the environment outside of the chamber 414 through the opening 416. In an embodiment, the porous material 415 may be configured to wick any bodily fluids away from the opening 416, thereby preventing the bodily fluids from escaping the chamber 414.

The porous material 415 may be similar or identical to any of the porous materials disclosed herein in one or more aspects. The porous material 415 includes at least one nonwoven material, such as any of the nonwoven material disclosed herein. In an example, as illustrated, the porous material 415 includes a plurality of layers (e.g., at least a first layer 434 and a second layer 436) similar to the porous material 215 illustrated in FIG. 2. At least one of the plurality of layers of the porous material 415 includes a nonwoven material and, optionally, a remainder of the porous material 415 includes one or more woven materials. In an example, the porous material 415 may include a single layer of nonwoven material similar to the porous material 115 illustrated in FIG. 1B.

The porous material 415 may at least substantially completely fills the portions of the chamber 414 that are not occupied by the conduit 430. In some examples, the porous material 415 may not substantially completely fill the portions of the chamber 414 that are not occupied by the conduit 430. In such an example, the fluid collection assembly 400 includes the reservoir 458 disposed in the chamber 414.

The reservoir 458 is a substantially unoccupied portion of the chamber 414. The reservoir 458 may be defined between the fluid impermeable barrier 408 and the porous material 415. The bodily fluids that are in the chamber 414 may flow through the porous material 415 to the reservoir 458. The reservoir 458 may retain of the bodily fluids therein. The fluid impermeable barrier 408 may retain the bodily fluids in the reservoir 458. While depicted in the first end region 420, the reservoir 458 may be located in any portion of the chamber 414 such as the second end region 422. The reservoir 458 may be located in a portion of the chamber 414 that is designed to be located in a gravimetrically low point of the fluid collection assembly 400 when the fluid collection assembly 400 is worn.

In some examples (not shown), the fluid collection assembly 400 may include multiple reservoirs, such as a first reservoir that is located at the portion of the chamber 414 closest to the inlet of the conduit 430 (e.g., first end region 420) and a second reservoir that is located at the portion of the of the chamber 414 that is at or near second end region 422). In another example, the porous material 415 is spaced from at least a portion of the conduit 430, and the reservoir 458 may be the space between the porous material 415 and the conduit 430.

The conduit 430 may be at least partially disposed in the chamber 414. The conduit 430 may be used to remove the bodily fluids from the chamber 414. The conduit 430 (e.g., a tube) includes an inlet of the conduit 430 and an outlet 412 positioned downstream from the inlet of the conduit 430. The outlet 412 may be operably coupled to a suction source, such as a vacuum pump for withdrawing fluid from the chamber 414 through the conduit 430. For example, the conduit 430 may extend into the fluid impermeable barrier 408 from the second end region 422 and may extend to the first end region 420 to a point proximate to the reservoir 458 therein such that the inlet of the conduit 430 is in fluid communication with the reservoir 458. The conduit 430 fluidly couples the chamber 414 with the fluid storage container (not shown) or the vacuum source (not shown).

The conduit 430 may extend through a bore in the porous material 415, such as into the reservoir 458. For example, the inlet of the conduit 430 may be extend into or be positioned in the reservoir 458. In the illustrated embodiment, the conduit 430 is at least partially disposed in the reservoir 458. In some examples (not shown), the conduit 430 may enter the chamber 414 in the distal end region and the inlet of the conduit 430 of the conduit 430 may be disposed in the distal end region (e.g., in the reservoir 458). The bodily fluids collected in the fluid collection assembly 400 may be removed from the chamber 414 via the conduit 430.

In some examples, the inlet of the conduit 430 may not extend into the reservoir 458. In such examples, the inlet of the conduit 430 may be disposed within the porous material 415 or at a terminal end thereof. For example, an end of the conduit 430 may be coextensive with or recessed within the porous material 415.

During use, the first end region 420 may be the gravimetrically low point of the chamber 414. As such, locating the inlet of the conduit 430 at or near a location expected to be the gravimetrically low point of the chamber 414 when worn by a patient enables the conduit 430 to receive more of the bodily fluids than if inlet of the conduit 430 was located elsewhere and reduce the likelihood of pooling (e.g., pooling of the bodily fluids may cause microbe growth and foul odors). For instance, as previously discussed, the bodily fluids in the porous material 415 may flow in any direction due to capillary forces. However, the bodily fluids may exhibit a preference to flow in the direction of gravity, especially when at least a portion of the porous material 415 is saturated with the bodily fluids. Accordingly, one or more of the inlet of the conduit 430 or the reservoir 458 may be located in the fluid collection assembly 400 in a position expected to be the gravimetrically low point in the fluid collection assembly 400 when worn by a patient, such as the first end region 420.

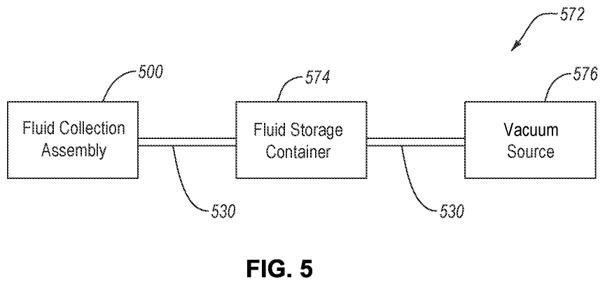

Other embodiments of fluid impermeable barriers, porous materials, chambers, and their shapes and configurations are disclosed in U.S. patent application Ser. No. 15/612,325 filed on Jun. 2, 2017; U.S. patent application Ser. No. 15/260,103 filed on Sep. 8, 2016; and U.S. Pat. No. 10,390,989 filed on Sep. 8, 2016, the disclosure of each of which is incorporated herein, in its entirety, by this reference. FIG. 5 is a block diagram of a system 572 for fluid collection, according to an embodiment. The system 572 includes a fluid collection assembly 500, a fluid storage container 574, and a vacuum source 576. The fluid collection assembly 500, the fluid storage container 574, and the vacuum source 576 may be fluidly coupled to each other via one or more conduits 530. For example, fluid collection assembly 500 may be operably coupled to one or more of the fluid storage container 574 or the vacuum source 576 via the conduit 530. Bodily fluids (e.g., urine or other bodily fluids) collected in the fluid collection assembly 500 may be removed from the fluid collection assembly 500 via the conduit 530 which is in fluid communication with the fluid collection assembly 500. For example, an inlet of the conduit 530 may extend into the fluid collection assembly 500, such as to a reservoir therein. The outlet of the conduit 530 may extend into the fluid storage container 574 or the vacuum source 576. The vacuum source 574 may provide a vacuum pressure to the chamber of the fluid collection assembly 500 via the conduit 530.

The vacuum pressure may be applied to the chamber of the fluid collection assembly 500 by the vacuum source 576 either directly or indirectly. The vacuum pressure may be applied indirectly via the fluid storage container 574. For example, the outlet of the conduit 530 may be disposed within the fluid storage container 574 and an additional conduit 530 may extend from the fluid storage container 574 to the vacuum source 576. Accordingly, the vacuum source 576 may apply the vacuum pressure to the fluid collection assembly 500 via the fluid storage container 574. The vacuum pressure may be applied directly via the vacuum source 576. For example, the outlet of the conduit 530 may be disposed within the vacuum source 576. An additional conduit 530 may extend from the vacuum source 576 to a point outside of the fluid collection assembly 500, such as to the fluid storage container 574. In such examples, the vacuum source 576 may be disposed between the fluid collection assembly 500 and the fluid storage container 574.

The fluid collection assembly 500 may be similar or identical to any of the fluid collection assemblies disclosed herein in one or more aspects. The fluid collection assembly 500 may be shaped and sized to be positioned adjacent to a female urethral opening or have a male urethral opening positioned therethrough (e.g., receive a penis therein). For example, the fluid collection assembly 500 may include a fluid impermeable barrier at least partially defining a chamber (e.g., interior region) of the fluid collection assembly 500. The fluid impermeable barrier also defines at least one opening extending therethrough from the external environment. The opening may be positioned adjacent to a female urethral opening or have a male urethral opening positioned therethrough. The fluid collection assembly 500 may include porous material disposed in the chamber. The porous material of the fluid collection assembly 500 is the same or substantially similar to any of the porous materials disclosed herein. For example, the porous material includes at least one nonwoven material.

The fluid storage container 574 is sized and shaped to retain the bodily fluids therein. The fluid storage container 574 may include a bag (e.g., drainage bag), a bottle or cup (e.g., collection jar), or any other enclosed container for storing bodily fluids such as urine. In some examples, the conduit 530 may extend from the fluid collection assembly 500 and attach to the fluid storage container 574 at a first point therein. An additional conduit 530 may attach to the fluid storage container 574 at a second point thereon and may extend and attach to the vacuum source 576. Accordingly, a vacuum (e.g., suction) may be drawn through fluid collection assembly 500 via the fluid storage container 574. Fluid, such as urine, may be drained from the fluid collection assembly 500 using the vacuum source 576.

The vacuum source 576 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 576 may provide a vacuum pressure to remove fluid from the fluid collection assembly 500. In some examples, the vacuum source 576 may be powered by one or more of a power cord (e.g., connected to a power socket), one or more batteries, or even manual power (e.g., a hand operated vacuum pump). In some examples, the vacuum source 576 may be sized and shaped to fit outside of, on, or within the fluid collection assembly 500. For example, the vacuum source 576 may include one or more miniaturized pumps or one or more micro pumps. The vacuum sources 576 disclosed herein may include one or more of a switch, a button, a plug, a remote, or any other device suitable to activate the vacuum source 576.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean±10%, ±5%, or +2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A fluid collection assembly, comprising:
a fluid impermeable barrier at least defining:
a chamber;
at least one opening; and
a fluid outlet;
at least one porous material disposed in the chamber, the at least one porous material including at least one nonwoven material, the at least one nonwoven material exhibiting a length, a width that is smaller than the length, and a thickness that is smaller than the width, the thickness measured parallel to a vertical direction and the length measured parallel to a horizontal direction, wherein the at least one nonwoven material is configured to wick one or more bodily fluids received by the nonwoven material in the vertical and horizontal direction.

2. The fluid collection assembly of claim 1, wherein the width is measured in the horizontal direction.

3. The fluid collection assembly of claim 1, wherein the at least one nonwoven material includes at least one carded web.

4. The fluid collection assembly of claim 1, wherein the at least one nonwoven material includes at least one needle punched web.

5. The fluid collection assembly of claim 1, wherein the at least one nonwoven material further includes at least one air laid web.

6. The fluid collection assembly of claim 1, wherein the at least one nonwoven material includes at least one spunlace web.

7. The fluid collection assembly of claim 1, wherein the at least one nonwoven material includes at least one vertical lapped nonwoven fabric.

8. The fluid collection assembly of claim 1, wherein the at least one nonwoven material includes one or more of at least one horizontal lapped nonwoven fabric or at least one crossed lapped nonwoven fabric.

9. The fluid collection assembly of claim 1, wherein the at least one nonwoven material is configured to withstand a vacuum pressure of about 40 kPa or greater without collapsing.

10. The fluid collection assembly of claim 1, wherein the at least one nonwoven material is configured withstand a vacuum pressure of about 5 kPa to about 30 kPa without collapsing.

11. The fluid collection assembly of claim 1, wherein the at least one nonwoven material exhibits a density of about 15 kg/m³ to about 30 kg/m³.

12. The fluid collection assembly of claim 1, wherein the at least one nonwoven material exhibits a length, a width that is smaller than the length, and a thickness that is smaller than the width, and wherein the thickness is about 3 mm to about 15 mm.

13. The fluid collection assembly of claim 1, wherein the at least one nonwoven material exhibits a basis weight of about 100 g/m² to about 250 g/m².

14. The fluid collection assembly of claim 1, wherein the at least one nonwoven material is hydrophilic.

15. The fluid collection assembly of claim 1, wherein the at least one nonwoven material is formed from a plurality of hollow fibers.

16. The fluid collection assembly of claim 1, wherein the at least one nonwoven material is formed from a plurality of fibers exhibiting an average length of about 1 cm or greater.

17. The fluid collection assembly of claim 1, wherein the at least one porous material includes a first layer and a second layer, wherein at least a portion of the first layer is configured to contact skin of the patient during use and at least a portion of the second layer is spaced from the skin of the patient by the at least a portion of the first layer during use, the first layer includes at least one woven material disposed and the second layer includes the at least one nonwoven material.

18. The fluid collection assembly of claim 1, wherein the chamber is configured to receive a penis.

19. The fluid collection assembly of claim 1, wherein the at least one porous material extends across the at least one opening, the portion of the at least one porous material that extends across the opening is configured to be positioned adjacent to a urethral opening.

20. A fluid collection system, comprising:
a fluid storage container configured to hold one or more bodily fluids therein;
a fluid collection assembly including:
a fluid impermeable barrier at least defining:
a chamber;
at least one opening; and
a fluid outlet;
at least one porous material disposed in the chamber, the at least one porous material including at least one nonwoven material, the at least one nonwoven material exhibiting a length, a width that is smaller than the length, and a thickness that is smaller than the width, the thickness measured parallel to a vertical direction and the length measured parallel to a horizontal direction, wherein the at least one nonwoven material is configured to wick one or more bodily fluids received by the nonwoven material in the vertical and horizontal direction; and
a vacuum source in fluid communication with the fluid storage container and the fluid collection assembly, the vacuum source configured to draw the one or more bodily fluids from the fluid collection assembly and deposit the one or more bodily fluids in the fluid storage container via one or more conduits.
